(19) United States
(12) Patent Application Publication
(10) Pub. No.: US 2019/0234962 A1
(43) Pub. Date: Aug. 1, 2019

(54) ENTROPY OF IMMUNE HEALTH

(71) Applicant: ARIZONA BOARD OF REGENTS ON BEHALF OF ARIZONA STATE UNIVERSITY, Scottsdale, AZ (US)

(72) Inventors: Stephen Johnston, Tempe, AZ (US); Kurt Whittemore, Cedar City, UT (US); Phillip Stafford, Phoenix, AZ (US); Lu Wang, Tempe, AZ (US)

(21) Appl. No.: 16/116,592

(22) Filed: Aug. 29, 2018

Related U.S. Application Data
(60) Provisional application No. 62/553,002, filed on Aug. 31, 2017.

(51) Int. Cl. G01N 33/68; G16H 50/50; G16H 50/20; G16H 50/80

(52) U.S. Cl. CPC G01N 33/6854; G01N 33/6848; G16H 50/80; G16H 50/20; G16H 50/50

(57) ABSTRACT

In certain embodiments, the present invention provides methods and compositions to measure unbiasedly the immune health status of an individual or population. A number of measures, including Shannon's entropy, can provide a measure of the diversity and disorder in the population of antibodies in a subject. The measure can be established by reacting the population of antibodies in a subject's blood with a complex surface, such as a peptide array.

### CROSS-REFERENCE TO RELATED APPLICATION(S)

This patent application claims the benefit of priority of U.S. application Ser. No. 62/553,002, filed Aug. 31, 2017, which application is herein incorporated by reference.

### STATEMENT OF GOVERNMENT SUPPORT

This invention was made with government support under HSHQDC-15-C-B0008 awarded by the Department of Homeland Security, Science and Technology. The government has certain rights in the invention.

### BACKGROUND

The antibodies in an individual's blood offer a tremendously valuable source of information. The 10⁹ types in an individual, and 10¹² total variants, exist in widely different concentrations and affinities for their original targets. There are also five major isotypes adding to the richness of this information. Many strategies have been employed to decipher this complexity. Arrays of proteins representing some or all of the proteome of a species are produced commercially. These can be used to discover antibodies against pathogen proteins or autoantibodies. Peptide arrays representing the proteomes provide higher resolution for the antibody binding to known proteins. Random sequence peptides or any sufficiently complex surface can be used to display the complexity of the antibodies in a subject. Alternatively, high throughput sequencing can be used to read the total variable regions of B and T cells. The composite of all of the sequences represents the profile of the antibody coding regions for a particular sample. The complexity of the antibody repertoire represents the status of the immune health.

New methods and products are needed to determine immune health.

### SUMMARY

In certain embodiments, provided herein is a peptide array platform comprising 10⁴, 10⁵, 10⁶ or more peptides chosen from random sequence space and 6-20 amino acids long operably attached to a solid substrate having an area of about 0.5 cm².

In certain embodiments, provided herein is a method of measuring the immune health of a subject or population by quantifying the diversity, organization and disorder of the antibodies in the subject, the method comprising (a) contacting a physiological sample with an array platform comprising at least 10⁴ to 10⁸ peptides of random sequences, wherein each peptide is 6-20 amino acids long and is operably attached to a solid substrate having an area of about 0.5 cm² to 2.5 cm², to form a sample-coated array platform, (b) contacting the sample-coated array platform with a labeled binding agent that binds to the sample, and (c) measuring an intensity distribution of the label.

In certain embodiments, provided herein is a method for determining the complexity of a mixture of antibodies comprising (a) contacting a physiological sample (e.g., a blood, serum, plasma or saliva sample) with an array platform comprising 10⁴, 10⁵, 10⁶ or more peptides of random sequences, wherein each peptide is 6-20 amino acids long and is operably attached to a solid substrate having an area of about 0.5 cm² to 2.5 cm² with to form a sample-coated array platform, (b) contacting the sample-coated array platform with a labeled binding agent that binds to the sample, and (c) measuring an intensity distribution of the label.

In certain embodiments, provided herein is a method for determining Shannon immune entropy (IE) in an individual comprising (a) contacting a sample with an array platform comprising 10⁴, 10⁵, 10⁶ or more peptides of random sequences, wherein each peptide is 6-20 amino acids long operably attached to a solid substrate having an area of about 0.5 cm² to 2.5 cm² to form a sample-coated platform, (b) contacting the sample-coated array platform with a labeled binding agent that binds to the sample, (c) measuring an intensity distribution of the label, and (d) calculating the Shannon IE of feature fluorescence.

In certain embodiments, provided herein is a method for characterizing the binding distribution of an antibody or aptamer comprising: (a) contacting a physiological sample with an array platform comprising at least 10⁴ to 10⁸ peptides of random sequences, wherein each peptide is 6-20 amino acids long and is operably linked to a solid substrate having an area of about 0.5 cm² to 2.5 cm² to form a sample-coated array platform, (b) contacting the sample-coated array platform with a labeled binding agent that binds to the sample, and (c) measuring an intensity distribution of the label.

In certain embodiments, provided herein is a method for determining a difference in distribution of two immune entropy (IE) datasets relating to a subject comprising (a) calculating a first IE dataset value for an individual using the method described herein, (b) calculating a second IE dataset value for the individual using the method described herein, and (c) determining the change in IE dataset values.

In certain embodiments, provided herein is a method of treating a subject with modified IE by indicating further diagnostic analysis or administering a therapeutic agent to the patient.

In certain embodiments, provided herein is a method of determining an IE value comprising (a) applying a sample to an array platform comprising 10⁴, 10⁵, 10⁶ or more peptides of random sequences, wherein each peptide is 6-20 amino acids long operably linked to a solid substrate having an area of about 0.5 cm² to 2.5 cm², (b) pre-washing the platform in 10% acetonitrile, 1% BSA to remove unbound peptides, (c) blocking the platform with 1×PBS pH 7.3, 3% BSA, 0.05% Tween 20, 0.014% β-mercaptohexanol, (d) immersing the platform in sample buffer consisting of 3% BSA, 1×PBS, and 0.05% Tween 20 pH 7.2, (e) diluting a subject's serum sample at least 1:500 and applying the diluted sample to the platform, (f) washing the platform in 1× Tris-buffered saline with 0.05% Tween 20 (TBST) pH 7.2, (g) applying to the platform an anti-human secondary antibody conjugated to a dye, (h) washing the platform, and (i) scanning the platform to determine the intensity of the dye.

In certain embodiments, provided herein is a method of determining an IE value comprising (a) loading a platform comprising at least 10⁴ to 10⁸ peptides of random sequences, wherein each peptide is 6-20 amino acids long and is operably linked to a well in a multi-well Array-It gasket, (b) adding a volume of 100 μl of incubation buffer to each well in the platform, (c) diluting a subject's serum sample at least 1:500 and applying the diluted sample to the plurality of wells in the platform, (d) washing the platform with PBST using a BioTek 405TS plate washer, (e) applying to the plurality of wells in the platform an anti-human secondary antibody conjugated to a dye, (f) washing the platform, and (g) scanning the platform to determine the intensity of the dye. In certain embodiments, provided herein is a method of monitoring a population for disease outbreak comprising (a) determining a first IE value of a plurality of individuals in a population at a first time point using the method described herein, (b) determining a second IE value of a plurality of individuals in a population at a second time point, and (c) comparing the first and second IE values to determine the change in immune entropy.

### BRIEF DESCRIPTION OF THE FIGURES

FIG. 1. Entropy measurement is able to distinguish a single monoclonal antibody profile from a mixed monoclonal profile. Antibody1 and antibody 2 are individually applied to the Immunosignature platform and then mixed together to apply for the Immunosignature platform. The entropy value is calculated for each distribution. The two monoclonal antibody entropies cannot be differentiated, while both of them are obviously lower than mixing the two antibodies together.

FIG. 2. Entropy measurement variance by different factors. Entropy value was tested with factors of age, gender, location (state), ethnicity and blood type. Age, gender, and location are found not to influence the entropy value, while ethnicity and blood type has significant influence on the entropy value. The p-value is obtained from an ANOVA test for each comparison.

FIGS. 3A-3C. Entropy measurement variance between individuals over time and with changes in health states. (FIG. 3A), boxplot of 5 individual's entropy recorded over a period of time shows difference from person to person. (FIG. 3B), plotting entropy against time for the volunteers shows variation of entropy that is independent between individuals. (FIG. 3C), recorded volunteer's activity shows entropy changes with vaccine administration and sickness. Black dots are blood draw points and the red line connects the dots.

FIG. 4. People recovering from infectious diseases have a higher entropy values compared with normal donors. Samples from seven types of infections are mixed together to represent the disease group. A t-test shows that the entropy from the disease group is significantly higher compared with the normal donors. P-value <0.0044.

FIGS. 5A-5B. Comparison of cancer patients with normal donors. (FIG. 5A) Various cancer samples are used to represent the general cancer group. The boxplot shows that cancer samples have a higher entropy value compared with normal donors by t-Test with p-value<0.0007. (FIG. 5B)

FIGS. 6A-6B. Example of entropy measuring the difference in an information distribution. (FIG. 6A) is the letter distribution (Wang et al., Scientific Reports, 7, Article number: 18060 (2017). (FIG. 6B) is the letter distribution of randomly generated thesis with the same total number of letters. The selective use of words results in order for the distribution. The outcome is that the normalized entropy is lower in the real dissertation than the randomly generated one, 0.887 compared with 1.

FIG. 7. Infections listed individually and in comparison with normal donors. The overall p-value from AVONA test is not significant from this comparison. Six of the seven infections have higher mean entropy than normal donors.

FIG. 8. Entropy record of one individual at different time points. The volunteer is healthy at the first five data points but report unknown illness at T6. Dramatic increase is observed at T6.

FIG. 9. Overview of 200 volunteers across eight timepoints with blood donated once per week. In the heatmap, every volunteer is shown as eight different samples (columns) and 50,000 of the 125,000 peptides from the immunosignature array displayed (rows). In every case, each person's eight timepoints cluster together. This information is displayed as a contrast to the Entropy scores, shown in subsequent figures.

FIG. 10. When immunosignature data is compressed into single values using Shannon's Entropy, the resulting values can illustrate patterns. Here all volunteers' Entropy scores are sorted by date, from earliest (left) to latest (right). In this example, there are two notable drops in the population entropy, the first starting at point 300, the second starting at point 1180. These timepoints shared two common features, the first time occurred immediately after the seasonal holiday in December, the second just after the Spring semester at Arizona State University completed. This illustrates that IE scores can display patterns in populations.

FIG. 11. Entropy scores can be further analyzed for population effects. In the heatmap on the left, individual Entropy scores are listed per volunteer (columns) over time, the earliest score listed on the left, the latest on the right. The change in Entropy over time, the average score, and the variance from point to point contribute to values that can be clustered, resulting in groups of volunteers that share similar characteristics over the duration of the study. On the right is a line-plot of the centers (mean) of each of the clusters identified on the left using k-means (k=12). The change in Entropy score over time is a definable characteristic of the population and can be used to extract information from volunteers using only Entropy scores as the basis.

### DETAILED DESCRIPTION

Immune Entropy (IE) Array Platform

In certain embodiments, provided herein is a measure of the organization (e.g., entropy) of the antibodies in a subject using a platform comprising 10⁴, 10⁵, 10⁶ or more peptides of random sequences that are 6-20 amino acids long and are operably attached to a solid substrate having an area of about 0.5 cm² to 2.5 cm².

In certain embodiments, the solid substrate is glass, silicone, quartz or other form of slide.

In certain embodiments, the solid substrate is glass. In certain embodiments, the glass is aminosilane-coated. In other embodiments, the glass is coated with nitrocellulose, epoxy, a dendrimer or other surface for attachment of peptides. In certain embodiments, the peptides are operably linked by means of maleimide conjugation to a linker, and wherein the linker is operably linked to the aminosilane-coated glass. The peptide could be linked to the N-terminal amine or C-terminal COOH.

In addition to the arrays and multi-well plates described above, in certain embodiments, other surfaces of sufficient complexity are used to determine the diversity in binding characteristics and amount of antibodies in a subject. For example, an array of natural proteins or peptides from them is used to splay out antibody diversity. In certain embodiments, the complex surface is composed of various glycoproteins, sugars, organic or inorganic chemicals. The only required for the surface is that it have sufficient chemical diversity and number to display the diversity in the antibody composition.

Methods of Determining Immune Entropy (IE)

In certain embodiments, provided herein is a method of determining an immune entropy (IE) comprising (a) contacting a physiological sample (e.g., a blood, serum, plasma or saliva sample) with a peptide array platform comprising 10⁴, 10⁵, 10⁶ or more peptides of random sequences and 6-20 amino acids long that are operably attached to a solid substrate having an area of about 0.5 cm² to 2.5 cm² to form a sample-coated array platform, (b) contacting the sample-coated array platform with a labeled binding agent that binds to the sample, and (c) measuring an intensity distribution of the label.

In certain embodiments, the labeled binding agent is a labeled antibody. In some embodiments, the agent is a synthetic antibody (i.e., “synbody”) or an aptamer.

In certain embodiments, the label is a fluorescent label. In some embodiments, the label is a quantum dot or gold nanosphere.

In some embodiments, the binding of the antibodies is directly measured by mass spectrometry or other label-free detection system.

In certain embodiments, provided herein is a method of determining an IE value comprising (a) loading a platform comprising a plurality of wells into a multi-well Array-It gasket, (b) adding a volume of 1-100 μl of incubation buffer to each well in the platform, (c) diluting a patient serum sample at 1:50, 1:100 or more and applying the diluted sample to the plurality of wells in the platform, (d) washing the platform with PBST using a BioTek 405TS plate washer, (e) applying to the plurality of wells in the platform an anti-human secondary antibody conjugated to a dye, (f) washing the platform, and (g) scanning the platform to determine the intensity of the dye.

In certain embodiments, provided herein is a method of determining an IE value comprising (a) applying a sample to an array platform comprising 10⁴, 10⁵, 10⁶ or more peptides of random sequences, wherein each peptide is 6-20 amino acids long and is operably attached to a solid substrate having an area of about 0.5 cm² up to 2.5 cm², (b) pre-washing the platform in 10% acetonitrile, 1% BSA to remove unbound peptides, (c) blocking the platform with 1×PBS pH 7.3, 3% BSA, 0.05% Tween 20, 0.014% β-mercaptohexanol, (d) immersing the platform in sample buffer consisting of 3% BSA, 1×PBS, and 0.05% Tween 20 pH 7.2, (e) diluting a patient serum sample at least 1:500 and applying the diluted sample to the platform, (f) washing the platform in 1×Tris-buffered saline with 0.05% Tween 20 (TBST) pH 7.2, (g) applying to the platform an anti-human secondary antibody conjugated to a dye, (h) washing the platform, and (i) laser scanning the platform or taking CCD image to determine the intensity of the dye.

In certain embodiments, in step (b) the platform is pre-washed with 10% acetonitrile, 1% BSA.

In certain embodiments, in step (c) the blocking solution is 1×PBS pH 7.3, 3% BSA, 0.05% Tween 20, 0.014% β-mercaptohexanol.

In certain embodiments, in step (d) the sample buffer comprises 3% BSA, 1×PBS, and 0.05% Tween 20 pH 7.2.

In certain embodiments, in step (f) the second wash solution comprises 1× Tris-buffered saline with 0.05% Tween 20 (TBST) pH 7.2.

In certain embodiments, solid substrate is glass, silicone, quartz or other form of slide.

In certain embodiments, the solid substrate is coated with aminosilane, nitrocellulose, epoxy, dendrimers, or other platform for attachment of peptides.

In certain embodiments, the peptides are operably linked by means of maleimide conjugation to a linker, and wherein the linker is operably linked to the aminosilane-coated glass.

Methods of Determining a Shannon Immune Entropy (IE) for an Individual

In certain embodiments, provided herein is a method for determining Shannon immune entropy (IE) in an individual comprising (a) contacting a physiological sample with an array platform comprising 10⁴, 10⁵, 10⁶ or more peptides of random sequences, wherein each peptide is 6-20 amino acids long and is operably attached to a solid substrate having an area of about 0.5 cm² to 2.5 cm² to form a sample-coated array platform, (b) contacting the sample-coated array platform with a labeled binding agent that binds to the sample, (c) measuring an intensity distribution of the label on the features, and (d) calculating the Shannon IE of peptide fluorescence, as described in the Example below.

In certain embodiments, provided herein is a method for determining a difference in distribution of two immune entropy (IE) datasets relating to a subject comprising (a) calculating a first IE dataset value for an individual using the method described herein, (b) calculating a second IE dataset value for the individual using the method described herein, and (c) determining the change in IE dataset values.

In certain embodiments, provided herein is a method for determining a difference in distribution of two immune entropy (IE) datasets relating to a patient comprising (a) calculating a first IE dataset value for an individual using the method described herein, (b) calculating a second IE dataset value for the individual using the method described herein, and (c) determining the change in IE dataset values.

In certain embodiments, the second data set is calculated from a sample taken from the patient at least one day later than the first sample.

In certain embodiments, the second data set is calculated from a sample taken from the patient at least one week later than the first sample.

In certain embodiments, the second data set is calculated from a sample taken from the patient at least one month later than the first sample.

In some embodiments the average or distribution of immune entropy is determined and compared on a population level for example to detect an outbreak of disease.

Other Forms of Immune Measures

Besides IE, there are other measures that can convey and compare the organization, complexity and disorder of the antibodies in an individual at a single time, over time or in a population. A list of such measures is given below. The unique feature is to use these commonly used calculations to convey the health and changes in a subject's immune system. In addition to Shannon's entropy, other methods include the following different measures of immune health:
normalized_entropy (normalized to rest of measured individuals)
cv (coefficient of variance of all peptides, normalized or raw)
stdev (standard deviation of all peptides, normalized or raw)
mean (mean of all peptides)
median (median of all peptides)
min (minimum value of all peptides)
max (maximum value of all peptides)
kurtosis (kurtosis of all peptides, normalized or raw—how sharp is the peak of the peptide distribution)
skew (how much the distribution, normalized or raw, is skewed left or right)
ninety_fifth_percentile (The upper 95th percentile, normalized or raw)
fifth_percentile (The lower 5th percentile, normalized or raw)
dynamic_range (The range of values from lowest to highest)
Sum of all log Ratios between all peptides (unordered)
Nonparametric sum of all log Ratios between all peptides (ordered)
log Ratio of every peptide pair combination (includes every possible combination once)
GOF=0 (how much the distribution, normalized or raw, varies from normal)
95th percentile dynamic range (The range between the upper and lower 5th percentile)

In certain embodiments, multiple assays are utilized, including the following forms of measurement of immune health:
Ordered ratio defined by peptides that change >1SD from time 1 to time 2, and time 2 to time 3, exclusively in one direction.
   Loess (parametric) 2nd order non-linear fit between timepoint 1 and timepoint 2.

Method of Using an Immune Entropy Value for a Population to Monitor for Disease Outbreak

A method of monitoring a population for disease outbreak comprising (a) determining a first IE value of a plurality of individuals in a population at a first time point using the method described herein, (b) determining a second IE value of a plurality of individuals in a population at a second time point, and (c) comparing the first and second IE values to determine the change in immune entropy. A large number of people having and increase or decrease in IE would indicate a population disturbance, regardless of the cause or causes. If the same features comprised the differences in IE it would indicate the disturbance had a common cause.

Labels and Methods of Detection

The detectable labels used in the methods can be primary labels (where the label comprises an element that is detected directly or that produces a directly detectable element) or secondary labels (where the detected label binds to a primary label, e.g., as is common in immunological labeling). An introduction to labels, labeling procedures and detection of labels is found in Polak and Van Noorden (1997) Introduction to Immunocytochemistry, 2nd ed., Springer Verlag, N.Y. and in Haugland (1996) Handbook of Fluorescent Probes and Research Chemicals, a combined handbook and catalogue Published by Molecular Probes, Inc., Eugene, Oreg. Patents that described the use of such labels include U.S. Pat. Nos. 3,817,837; 3,850,752; 3,939,350; 3,996,345; 4,277,437; 4,275,149; and 4,366,241.

Primary and secondary labels can include undetected elements as well as detected elements. Useful primary and secondary labels can include spectral labels such as green fluorescent protein, fluorescent dyes (e.g., fluorescein and derivatives such as fluorescein isothiocyanate (FITC) and Oregon Green™, rhodamine and derivatives (e.g., Texas red, tetrarhodimine isothiocynate (TRITC), etc.), digoxigenin, biotin, phycoerythrin, AMCA, CyDyes™, and the like), radiolabels (e.g., ³H, ¹²⁵I, ³⁵S, ¹⁴C, ³²P, ³³P, etc.), enzymes (e.g., horse radish peroxidase, alkaline phosphatase etc.), spectral calorimetric labels such as colloidal gold or colored glass or plastic (e.g. polystyrene, polypropylene, latex, etc.) beads. The label can be coupled directly or indirectly to a component of the detection assay (e.g., the detection reagent) according to methods well known in the art. As indicated above, a wide variety of labels may be used, with the choice of label depending on sensitivity required, ease of conjugation with the compound, stability requirements, available instrumentation, and disposal provisions.

Exemplary labels that can be used include those that use: 1) chemiluminescence (using horseradish peroxidase and/or alkaline phosphatase with substrates that produce photons as breakdown products as described above) with kits being available, e.g., from Molecular Probes, Amersham, Boehringer-Mannheim, and Life Technologies/Gibco BRL; 2) color production (using both horseradish peroxidase and/or alkaline phosphatase with substrates that produce a colored precipitate (kits available from Life Technologies/Gibco BRL, and Boehringer-Mannheim)); 3) fluorescence using, e.g., an enzyme such as alkaline phosphatase, together with the substrate AttoPhos (Amersham) or other substrates that produce fluorescent products, 4) fluorescence (e.g., using Cy-5 (Amersham), fluorescein, and other fluorescent tags); 5) radioactivity. Other methods for labeling and detection will be readily apparent to one skilled in the art.

The presence of a label can be detected by inspection, or a detector that monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising bound labeling moieties is digitized for subsequent computer analysis.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a blood (e.g., serum) sample, is generally washed to remove any non-specifically bound antibody species, allowing only those binding agents (e.g., antibodies) specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. Patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

After binding the random peptides to the array platform (e.g., wells), coating with a non-reactive material to reduce background, and washing to remove unbound material, the array platform is contacted with the biological sample to be tested under conditions effective to allow immune complex (peptide/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (peptide/antibody) formation” means that the conditions can include diluting the patient sample with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2-4 hours or so, at temperatures, e.g., on the order of 25° C. to 27° C., or may be overnight at about 4° C. or so.

Following all incubation steps in an ELISA, the contacted surface is washed so as to remove non-complexed material. An example of a washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. This may be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzothiazoline-6-sulfonic acid (ABTS), or H₂O₂, in the case of peroxidase as the enzyme label. Quantification is then achieved by measuring the degree of color generated, e.g., using a visible spectra spectrophotometer.

Antibodies and Antibody Fragments

IE can be applied to characterize single antibodies or libraries of antibodies. As used herein, the term “antibody” includes synthetic antibodies (synbodies), scFv, humanized, fully human or chimeric antibodies, single-chain antibodies, diabodies, and antigen-binding fragments of antibodies that do not contain the Fc region (e.g., Fab fragments). In certain embodiments, the antibody is a human antibody or a humanized antibody. A “humanized” antibody contains only the three CDRs (complementarity determining regions) and sometimes a few carefully selected “framework” residues (the non-CDR portions of the variable regions) from each donor antibody variable region recombinantly linked onto the corresponding frameworks and constant regions of a human antibody sequence. A “fully humanized antibody” is created in a hybridoma from mice genetically engineered to have only human-derived antibody genes or by selection from a phage-display library of human-derived antibody genes.

As used herein, the term “antibody” includes a single-chain variable fragment (scFv or “nanobody”), humanized, fully human or chimeric antibodies, single-chain antibodies, diabodies, and antigen-binding fragments of antibodies (e.g., Fab fragments). A scFv is a fusion protein of the variable region of the heavy (V_{H}) and light chains (V_{L}) of an immunoglobulin that is connected by means of a linker peptide. The linker is usually short, about 10-25 amino acids in length. If flexibility is important, the linker will contain a significant number of glycines. If solubility is important, serines or theonines will be utilized in the linker. The linker may link the amino-terminus of the V_{H} to the carboxy-terminus of the V_{L}, or the linker may link the carboxy-terminus of the V_{H} to the amino-terminus of the V_{L}.

As used herein, the term “monoclonal antibody” refers to an antibody obtained from a group of substantially homogeneous antibodies, that is, an antibody group wherein the antibodies constituting the group are homogeneous except for naturally occurring mutants that exist in a small amount. Monoclonal antibodies are highly specific and interact with a single antigenic site. Furthermore, each monoclonal antibody targets a single antigenic determinant (epitope) on an antigen, as compared to common polyclonal antibody preparations that typically contain various antibodies against diverse antigenic determinants. In addition to their specificity, monoclonal antibodies are advantageous in that they are produced from hybridoma cultures not contaminated with other immunoglobulins.

The adjective “monoclonal” indicates a characteristic of antibodies obtained from a substantially homogeneous group of antibodies, and does not specify antibodies produced by a particular method. For example, a monoclonal antibody to be used can be produced by, for example, hybridoma methods (Kohler and Milstein, Nature 256:495, 1975) or recombination methods (U.S. Pat. No. 4,816,567). The monoclonal antibodies used can be also isolated from a phage antibody library (Clackson et al., Nature 352:624-628, 1991; Marks et al., J. Mol. Biol. 222:581-597, 1991). The monoclonal antibodies can include comprise “chimeric” antibodies (immunoglobulins), wherein a part of a heavy (H) chain and/or light (L) chain is derived from a specific species or a specific antibody class or subclass, and the remaining portion of the chain is derived from another species, or another antibody class or subclass. Furthermore, mutant antibodies and antibody fragments thereof are also included (U.S. Pat. No. 4,816,567; Morrison et al., Proc. Natl. Acad. Sci. USA 81:6851-6855, 1984).

As used herein, the term “mutant antibody” refers to an antibody comprising a variant amino acid sequence in which one or more amino acid residues have been altered. For example, the variable region of an antibody can be modified to improve its biological properties, such as antigen binding. Such modifications can be achieved by site-directed mutagenesis (see Kunkel, Proc. Natl. Acad. Sci. USA 82: 488 (1985)), PCR-based mutagenesis, cassette mutagenesis, and the like. Such mutants comprise an amino acid sequence which is at least 70% identical to the amino acid sequence of a heavy or light chain variable region of the antibody, e.g., at least 75%, e.g., at least 80%, e.g., at least 85%, e.g., at least 90%, e.g., at least 95% identical. As used herein, the term “sequence identity” is defined as the percentage of residues identical to those in the antibody's original amino acid sequence, determined after the sequences are aligned and gaps are appropriately introduced to maximize the sequence identity as necessary.

Specifically, the identity of one nucleotide sequence or amino acid sequence to another can be determined using the algorithm BLAST, by Karlin and Altschul (Proc. Natl. Acad. Sci. USA, 90: 5873-5877, 1993). Programs such as BLASTN and BLASTX were developed based on this algorithm (Altschul et al., J. Mol. Biol. 215: 403-410, 1990). To analyze nucleotide sequences according to BLASTN based on BLAST, the parameters are set, for example, as score=100 and wordlength=12. On the other hand, parameters used for the analysis of amino acid sequences by BLASTX based on BLAST include, for example, score=50 and wordlength=3. Default parameters for each program are used when using the BLAST and Gapped BLAST programs. Specific techniques for such analyses are known in the art (see the website of the National Center for Biotechnology Information (NCBI), Basic Local Alignment Search Tool (BLAST); http://www.ncbi.nlm.nih.gov).

Polyclonal and monoclonal antibodies can be prepared by methods known to those skilled in the art.

In another embodiment, antibodies or antibody fragments can be isolated from an antibody phage library, produced by using the technique reported by McCafferty et al. (Nature 348:552-554 (1990)). Clackson et al. (Nature 352:624-628 (1991)) and Marks et al. (J. Mol. Biol. 222:581-597 (1991)) reported on the respective isolation of mouse and human antibodies from phage libraries. There are also reports that describe the production of high affinity (nM range) human antibodies based on chain shuffling (Marks et al., Bio/Technology 10:779-783 (1992)), and combinatorial infection and in vivo recombination, which are methods for constructing large-scale phage libraries (Waterhouse et al., Nucleic Acids Res. 21:2265-2266 (1993)). These technologies can also be used to isolate monoclonal antibodies, instead of using conventional hybridoma technology for monoclonal antibody production.

Antibodies can be purified by a method appropriately selected from known methods, such as the protein A-Sepharose method, hydroxyapatite chromatography, salting-out method with sulfate, ion exchange chromatography, and affinity chromatography, or by the combined use of the same.

Recombinant antibodies, produced by gene engineering, may be used. The genes encoding the antibodies obtained by a method described above are isolated from the hybridomas. The genes are inserted into an appropriate vector, and then introduced into a host (see, e.g., Carl, A. K. Borrebaeck, James, W. Larrick, Therapeutic Monoclonal Antibodies, Published in the United Kingdom by Macmillan Publishers Ltd, 1990). The use of nucleic acids encoding the antibodies, and vectors comprising these nucleic acids, is also included. Specifically, using a reverse transcriptase, cDNAs encoding the variable regions (V regions) of the antibodies are synthesized from the mRNAs of hybridomas. After obtaining the DNAs encoding the variable regions of antibodies of interest, they are ligated with DNAs encoding desired constant regions (C regions) of the antibodies, and the resulting DNA constructs are inserted into expression vectors. Alternatively, the DNAs encoding the variable regions of the antibodies may be inserted into expression vectors comprising the DNAs of the antibody C regions. These are inserted into expression vectors so that the genes are expressed under the regulation of an expression regulatory region, for example, an enhancer and promoter. Then, host cells are transformed with the expression vectors to express the antibodies. Cells expressing antibodies are provided. The cells expressing antibodies include cells and hybridomas transformed with a gene of such an antibody.

The antibodies also include antibodies which comprise complementarity-determining regions (CDRs), or regions functionally equivalent to CDRs. The term “functionally equivalent” refers to comprising amino acid sequences similar to the amino acid sequences of CDRs of any of the monoclonal antibodies isolated in the Examples. The term “CDR” refers to a region in an antibody variable region (also called “V region”), and determines the specificity of antigen binding. The H chain and L chain each have three CDRs, designated from the N terminus as CDR1, CDR2, and CDR3. There are four regions flanking these CDRs: these regions are referred to as “framework,” and their amino acid sequences are highly conserved. The CDRs can be transplanted into other antibodies, and thus a recombinant antibody can be prepared by combining CDRs with the framework of a desired antibody. One or more amino acids of a CDR can be modified without losing the ability to bind to its antigen. For example, one or more amino acids in a CDR can be substituted, deleted, and/or added.

In certain embodiments, an amino acid residue is mutated into one that allows the properties of the amino acid side-chain to be conserved. Examples of the properties of amino acid side chains comprise: hydrophobic amino acids (A, I, L, M, F, P, W, Y, V), hydrophilic amino acids (R, D, N, C, E, Q, G, H, K, S, T), and amino acids comprising the following side chains: aliphatic side-chains (G, A, V, L, I, P); hydroxyl group-containing side-chains (S, T, Y); sulfur atom-containing side-chains (C, M); carboxylic acid- and amide-containing side-chains (D, N, E, Q); base-containing side-chains (R, K, H); and aromatic-containing side-chains (H, F, Y, W). The letters within parenthesis indicate the one-letter amino acid codes. Amino acid substitutions within each group are called conservative substitutions. It is well known that a polypeptide comprising a modified amino acid sequence in which one or more amino acid residues is deleted, added, and/or substituted can retain the original biological activity (Mark D. F. et al., Proc. Natl. Acad. Sci. U.S.A. 81:5662-5666 (1984); Zoller M. J. and Smith M., Nucleic Acids Res. 10: 6487-6500 (1982); Wang A. et al., Science 224: 1431-1433; Dalbadie-McFarland G. et al., Proc. Natl. Acad. Sci. U.S.A. 79: 6409-6413 (1982)). The number of mutated amino acids is not limited, but in general, the number falls within 40% of amino acids of each CDR, and e.g., within 35%, e.g., within 30% (e.g., within 25%). The identity of amino acid sequences can be determined as described herein.

Recombinant antibodies artificially modified to reduce heterologous antigenicity against humans can be used. Examples include chimeric antibodies and humanized antibodies. These modified antibodies can be produced using known methods. A chimeric antibody includes an antibody comprising variable and constant regions of species that are different to each other, for example, an antibody comprising the antibody heavy chain and light chain variable regions of a nonhuman mammal such as a mouse, and the antibody heavy chain and light chain constant regions of a human. Such an antibody can be obtained by (1) ligating a DNA encoding a variable region of a mouse antibody to a DNA encoding a constant region of a human antibody; (2) incorporating this into an expression vector; and (3) introducing the vector into a host for production of the antibody.

A humanized antibody, which is also called a reshaped human antibody, is obtained by substituting an H or L chain complementarity determining region (CDR) of an antibody of a nonhuman mammal such as a mouse, with the CDR of a human antibody. Conventional genetic recombination techniques for the preparation of such antibodies are known (see, for example, Jones et al., Nature 321: 522-525 (1986); Reichmann et al., Nature 332: 323-329 (1988); Presta Curr. Op. Struct. Biol. 2: 593-596 (1992)). Specifically, a DNA sequence designed to ligate a CDR of a mouse antibody with the framework regions (FRs) of a human antibody is synthesized by PCR, using several oligonucleotides constructed to comprise overlapping portions at their ends. A humanized antibody can be obtained by (1) ligating the resulting DNA to a DNA that encodes a human antibody constant region; (2) incorporating this into an expression vector; and (3) transfecting the vector into a host to produce the antibody (see, European Patent Application No. EP 239,400, and International Patent Application No. WO 96/02576). Human antibody FRs that are ligated via the CDR are selected where the CDR forms a favorable antigen-binding site. The humanized antibody may comprise additional amino acid residue(s) that are not included in the CDRs introduced into the recipient antibody, nor in the framework sequences. Such amino acid residues are usually introduced to more accurately optimize the antibody's ability to recognize and bind to an antigen. For example, as necessary, amino acids in the framework region of an antibody variable region may be substituted such that the CDR of a reshaped human antibody forms an appropriate antigen-binding site (Sato, K. et al., Cancer Res. (1993) 53, 851-856).

The isotypes of the antibodies are not limited. The isotypes include, for example, IgG (IgG1, IgG2, IgG3, and IgG4), IgM, IgA (IgA1 and IgA2), IgD, and IgE. The antibodies may also be antibody fragments comprising a portion responsible for antigen binding, or a modified fragment thereof. The term “antibody fragment” refers to a portion of a full-length antibody, and generally to a fragment comprising an antigen-binding domain or a variable region. Such antibody fragments include, for example, Fab, F(ab′)₂, Fv, single-chain Fv (scFv) which comprises a heavy chain Fv and a light chain Fv coupled together with an appropriate linker, diabody (diabodies), linear antibodies, and multispecific antibodies prepared from antibody fragments. Previously, antibody fragments were produced by digesting natural antibodies with a protease; currently, methods for expressing them as recombinant antibodies using genetic engineering techniques are also known (see Morimoto et al., Journal of Biochemical and Biophysical Methods 24:107-117 (1992); Brennan et al., Science 229:81 (1985); Co, M. S. et al., J. Immunol., 1994, 152, 2968-2976; Better, M. & Horwitz, A. H., Methods in Enzymology, 1989, 178, 476-496, Academic Press, Inc.; Plueckthun, A. & Skerra, A., Methods in Enzymology, 1989, 178, 476-496, Academic Press, Inc.; Lamoyi, E., Methods in Enzymology, 1989, 121, 663-669; Bird, R. E. et al., TIBTECH, 1991, 9, 132-137).

An “Fv” fragment is the smallest antibody fragment, and contains a complete antigen recognition site and a binding site. This region is a dimer (V_{H}-V_{L} dimer) wherein the variable regions of each of the heavy chain and light chain are strongly connected by a noncovalent bond. The three CDRs of each of the variable regions interact with each other to form an antigen-binding site on the surface of the V_{H}-V_{L} dimer. In other words, a total of six CDRs from the heavy and light chains function together as an antibody's antigen-binding site. However, a variable region (or a half Fv, which contains only three antigen-specific CDRS) alone is also known to be able to recognize and bind to an antigen, although its affinity is lower than the affinity of the entire binding site. Thus, an antibody fragment is an Fv fragment, but is not limited thereto. Such an antibody fragment may be a polypeptide which comprises an antibody fragment of heavy or light chain CDRs which are conserved, and which can recognize and bind its antigen.

A Fab fragment (also referred to as F(ab)) also contains a light chain constant region and heavy chain constant region (CH1). For example, papain digestion of an antibody produces the two kinds of fragments: an antigen-binding fragment, called a Fab fragment, containing the variable regions of a heavy chain and light chain, which serve as a single antigen-binding domain; and the remaining portion, which is called an “Fc” because it is readily crystallized. A Fab′ fragment is different from a Fab fragment in that a Fab′ fragment also has several residues derived from the carboxyl terminus of a heavy chain CH1 region, which contains one or more cysteine residues from the hinge region of an antibody. A Fab′ fragment is, however, structurally equivalent to Fab in that both are antigen-binding fragments which comprise the variable regions of a heavy chain and light chain, which serve as a single antigen-binding domain. Herein, an antigen-binding fragment comprising the variable regions of a heavy chain and light chain which serve as a single antigen-binding domain, and which is equivalent to that obtained by papain digestion, is referred to as a “Fab-like antibody,” even when it is not identical to an antibody fragment produced by protease digestion. Fab′-SH is Fab′ with one or more cysteine residues having free thiol groups in its constant region. A F(ab′) fragment is produced by cleaving the disulfide bond between the cysteine residues in the hinge region of F(ab′)₂. Other chemically crosslinked antibody fragments are also known to those skilled in the art. Pepsin digestion of an antibody yields two fragments; one is a F(ab′)₂ fragment which comprises two antigen-binding domains and can cross-react with antigens, and the other is the remaining fragment (referred to as pFc′). Herein, an antibody fragment equivalent to that obtained by pepsin digestion is referred to as a “F(ab′)₂-like antibody” when it comprises two antigen-binding domains and can cross-react with antigens. Such antibody fragments can also be produced, for example, by genetic engineering. Such antibody fragments can also be isolated, for example, from the antibody phage library described above. Alternatively, F(ab′)₂-SH fragments can be recovered directly from hosts, such as E. coli, and then allowed to form F(ab′)₂ fragments by chemical crosslinking (Carter et al., Bio/Technology 10:163-167 (1992)). In an alternative method, F(ab′)₂ fragments can be isolated directly from a culture of recombinant hosts.

A single-chain antibody (also referred to as “scFv”) can be prepared by linking a heavy chain V region and a light chain V region of an antibody (for a review of scFv see Pluckthun “The Pharmacology of Monoclonal Antibodies” Vol. 113, eds. Rosenburg and Moore, Springer Verlag, N.Y., pp. 269-315 (1994)). Methods for preparing single-chain antibodies are known in the art (see, for example, U.S. Pat. Nos. 4,946,778; 5,260,203; 5,091,513; and 5,455,030). In such scFvs, the heavy chain V region and the light chain V region are linked together via a linker, e.g., a polypeptide linker (Huston, J. S. et al., Proc. Natl. Acad. Sci. U.S.A., 1988, 85, 5879-5883). The heavy chain V region and the light chain V region in a scFv may be derived from the same antibody, or from different antibodies. The peptide linker used to ligate the V regions may be any single-chain peptide consisting of 12 to 19 residues. A DNA encoding a scFv can be amplified by PCR using, as a template, either the entire DNA, or a partial DNA encoding a desired amino acid sequence, selected from a DNA encoding the heavy chain or the V region of the heavy chain of the above antibody, and a DNA encoding the light chain or the V region of the light chain of the above antibody; and using a primer pair that defines the two ends. Further amplification can be subsequently conducted using a combination of the DNA encoding the peptide linker portion, and the primer pair that defines both ends of the DNA to be ligated to the heavy and light chain respectively. After constructing DNAs encoding scFvs, conventional methods can be used to obtain expression vectors comprising these DNAs, and hosts transformed by these expression vectors. Furthermore, scFvs can be obtained according to conventional methods using the resulting hosts. These antibody fragments can be produced in hosts by obtaining genes that encode the antibody fragments and expressing these as outlined above. Antibodies bound to various types of molecules, such as polyethylene glycols (PEGs), may be used as modified antibodies. Methods for modifying antibodies are already established in the art. The term “antibody” also encompasses the above-described antibodies.

The antibodies obtained can be purified to homogeneity. The antibodies can be isolated and purified by a method routinely used to isolate and purify proteins. The antibodies can be isolated and purified by the combined use of one or more methods appropriately selected from column chromatography, filtration, ultrafiltration, salting out, dialysis, preparative polyacrylamide gel electrophoresis, and isoelectro-focusing, for example (Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory, 1988). Such methods are not limited to those listed above. Chromatographic methods include affinity chromatography, ion exchange chromatography, hydrophobic chromatography, gel filtration, reverse-phase chromatography, and adsorption chromatography. These chromatographic methods can be practiced using liquid phase chromatography, such as HPLC and FPLC. Columns to be used in affinity chromatography include protein A columns and protein G columns. For example, protein A columns include Hyper D, POROS, and Sepharose F. F. (Pharmacia). Antibodies can also be purified by utilizing antigen binding, using carriers on which antigens have been immobilized.

The antibodies can be formulated according to standard methods (see, for example, Remington's Pharmaceutical Science, latest edition, Mark Publishing Company, Easton, U.S.A.), and may comprise pharmaceutically acceptable carriers and/or additives. Compositions (including reagents and pharmaceuticals) comprising the antibodies, and pharmaceutically acceptable carriers and/or additives, are also included. Exemplary carriers include surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, and corrigents. However, the carriers that may be employed are not limited to this list. In fact, other commonly used carriers can be appropriately employed: light anhydrous silicic acid, lactose, crystalline cellulose, mannitol, starch, carmelose calcium, carmelose sodium, hydroxypropylcellulose, hydroxypropylmethyl cellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, inorganic salt, and so on. The composition may also comprise other low-molecular-weight polypeptides, proteins such as serum albumin, gelatin, and immunoglobulin, and amino acids such as glycine, glutamine, asparagine, arginine, and lysine. When the composition is prepared as an aqueous solution for injection, it can comprise an isotonic solution comprising, for example, physiological saline, dextrose, and other adjuvants, including, for example, D-sorbitol, D-mannose, D-mannitol, and sodium chloride, which can also contain an appropriate solubilizing agent, for example, alcohol (for example, ethanol), polyalcohol (for example, propylene glycol and PEG), and non-ionic detergent (polysorbate 80 and HCO-50).

If necessary, antibodies may be encapsulated in microcapsules (microcapsules made of hydroxycellulose, gelatin, polymethylmethacrylate, and the like), and made into components of colloidal drug delivery systems (liposomes, albumin microspheres, microemulsions, nano-particles, and nano-capsules) (for example, see “Remington's Pharmaceutical Science 16th edition”, Oslo Ed. (1980)). Moreover, methods for making sustained-release drugs are known, and these can be applied for the antibodies (Langer et al., J. Biomed. Mater. Res. 15: 167-277 (1981); Langer, Chem. Tech. 12: 98-105 (1982); U.S. Pat. No. 3,773,919; EP Patent Application No. 58,481; Sidman et al., Biopolymers 22: 547-556 (1983); EP: 133,988).

Aptamers

Besides antibodies, the immune entropy of aptamers or libraries of aptamers can be measured. Aptamers are single stranded oligonucleotides that can naturally fold into different 3-dimensional structures, which have the capability of binding specifically to biosurfaces, a target compound or a moiety. The term “conformational change” refers to the process by which a nucleic acid, such as an aptamer, adopts a different secondary or tertiary structure. The term “fold” may be substituted for conformational change.

Aptamers have advantages over more traditional affinity molecules such as antibodies in that they are very stable, can be easily synthesized, and can be chemically manipulated with relative ease. Aptamer synthesis is potentially far cheaper and reproducible than antibody-based diagnostic tests. Aptamers are produced by solid phase chemical synthesis, an accurate and reproducible process with consistency among production batches. An aptamer can be produced in large quantities by polymerase chain reaction (PCR) and once the sequence is known, can be assembled from individual naturally occurring nucleotides and/or synthetic nucleotides. Aptamers are stable to long-term storage at room temperature, and, if denatured, aptamers can easily be renatured, a feature not shared by antibodies. Furthermore, aptamers have the potential to measure concentrations of ligand in orders of magnitude lower (parts per trillion or even quadrillion) than those antibody-based diagnostic tests. These characteristics of aptamers make them attractive for diagnostic applications.

Aptamers are typically oligonucleotides that may be single stranded oligodeoxynucleotides, oligoribonucleotides, or modified oligodeoxynucleotide or oligoribonucleotides. The term “modified” encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2-azido-ribose, carbocyclic sugar analogues a-anomeric sugars; epimeric sugars such as arabinose, xyloses or lyxoses, pyranose sugars, furanose sugars, and sedoheptulose.

Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4,N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methyl cytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2-methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6-diaminopurine; methylpsuedouracil; 1-methylguanine; 1-methylcytosine.

The aptamers can be synthesized using conventional phosphodiester linked nucleotides and synthesized using standard solid or solution phase synthesis techniques which are known in the art. Linkages between nucleotides may use alternative linking molecules. For example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—.

The invention will now be illustrated by the following non-limiting Examples.

### Example 1

### Entropy is a Simple Measure of the Antibody Profile and is an Indicator of Health Status

The IE technology is based on creating arrays of 10⁴, 10⁵, 10⁶ or more peptides, 6-20 amino acids long, in an area of ˜0.5 cm² to 2.5 cm². They are chosen from random peptide sequence space to optimize chemical diversity and therefore, presumably, binding distinctions between antibodies. Given that most epitopes of antibodies are 5-20aa long, it is unlikely that the exact cognate epitope for any antibody is present in the arrays. However, because of the avidity effect each antibody will bind many peptides in a characteristic signature. Therefore, when blood from an individual is applied, a complex pattern of antibody binding is produced unique for each sample. The binding varies in which features are bound and the amount of antibody on each feature. An attractive feature of IE is its simplicity. A drop of blood can be sent on a filter paper thru the mail, diluted and applied to the array to make the measurement, greatly facilitating monitoring individuals. Many other measures of entropy in biological systems (see below) are very complex to implement.

Here the information entropy of each sample is calculated. Shannon information entropy (defined as H=−Σp(x)*log(p(x)) where p(x) is the probability of outcome x) can be applied to any type of information to quantify how predictable the information is. In information theory, the entropy can be determined from the frequency of values for all of the elements contained in an object of information. For example, the entropy of the message “aaaa” would have a lower entropy value than the message “abcd”. The entropy value of the first message is −(4/4*log(4/4))=0, and the entropy of the second message is −(¼*log(¼)+¼*log(¼)+¼*log(¼)+¼*log(¼))=1.39. Therefore, high entropy information is most similar to the information that would be output by a random information generator.

Global measures, and the entropy measure in particular, have been applied to a variety of biological data previously. Global measures such as the mean and median of a sample are used extensively in scientific research. Application of information entropy is less common, but it has been used to characterize a wide range of different biological data. In cancer, the entropy calculated from aberrations in DNA copy number is higher in a variety of cancer types, alternative splicing entropy is higher in some cancers, the entropy of structural and numerical chromosomal aberrations is higher in cancers, the entropy of a random walk on the protein interaction network graph was higher in cancer cells, and the entropy of photographs of tissues was higher in cancer tissues. In the brain, the entropy of fMRI data increases with age and Alzheimer's disease in a dataset of 1,248 samples. Schizophrenic patients had a lower entropy value than normal subjects, which indicates that entropy values that are too low or too high may indicate that something is altered from normal in the system being investigated. Rhesus monkeys with induced Parkinson's disease had higher levels of neuronal firing entropy compared to controls. Entropy has also been used for data related to the immune system. For example, Vilar et al. assessed entropy from data sets on immune cells (Vilar, J. M. G. Entropy of Leukemia on Multidimensional Morphological and Molecular Landscapes. Physical Review X4, doi:10.1103/PhysRevX.4.021038 (2014)). Merilli et al. applied entropy values to the putative idiotypic network of antibodies (Rucco, M., Castiglione, F., Merelli, E. & Pettini, M. in Proceedings of ECCS 2014: European Conference on Complex Systems (eds Stefano Battiston, Francesco De Pellegrini, Guido Caldarelli, & Emanuela Merelli) 117-128 (Springer International Publishing, 2016)). Asti et al used maximum-entropy models based on antibody gene sequence data to predict antibody binding from complex mixtures (Asti, L., Uguzzoni, G., Marcatili, P. & Pagnani, A. Maximum-Entropy Models of Sequenced Immune Repertoires Predict Antigen-Antibody Affinity. PLoS computational biology 12, e1004870, doi:10.1371/journal.pcbi.1004870 (2016)).

Here the Shannon information entropy of the peptide fluorescence intensity distribution that results from applying sera to a complex peptide microarray surface is calculated. The immune entropy (IE) was measured in a wide array of people, the same people over time and the people with diseases. This simple approach to assign a single number for the health status of the immune system has many advantages for health monitoring of individuals or groups of people or animals.

Results

Entropy can Differentiate a Monoclonal Antibody Solution from a Mixed Antibody Solution

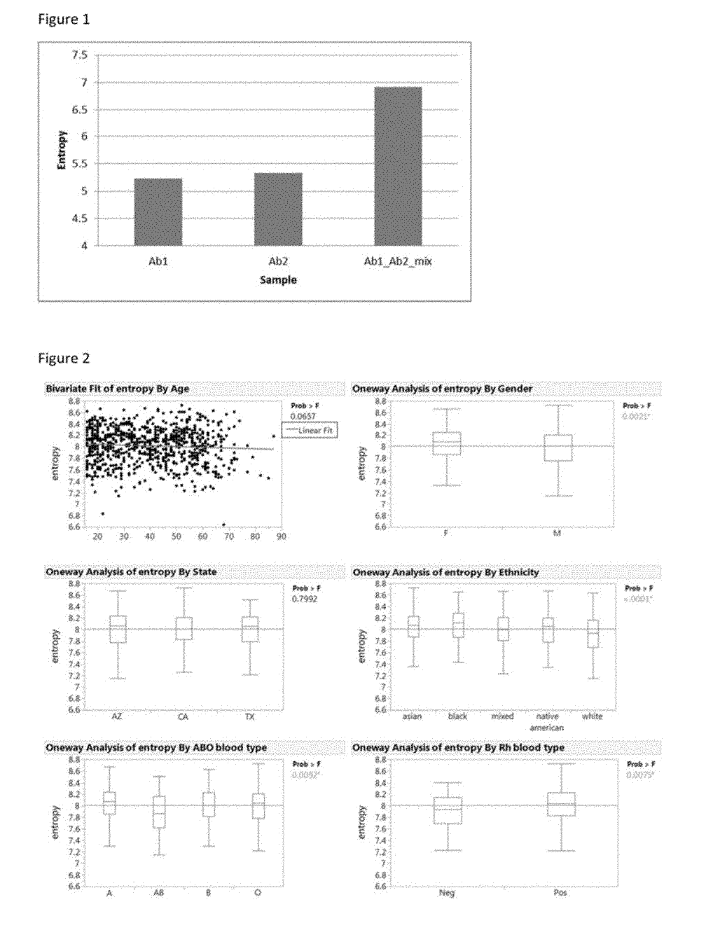

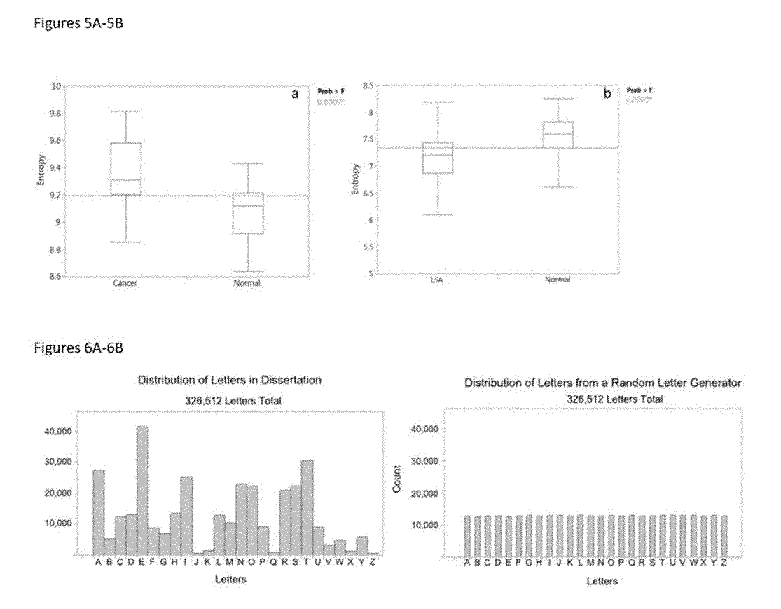

Entropy can generally measure the difference in the distribution of two datasets as illustrated by example in FIGS. 6A-6B. As applied to an IMS, the expectation is that more antibody types would produce more randomness, which should result in a higher entropy number. This hypothesis was tested by measuring the entropy of binding of two different monoclonal antibodies individually and then in an equal mixture. The results are shown in FIG. 1. The two monoclonals target different sites (RHSVV and SDLWKL) on the p53 protein. When each was applied separately to the array, they bound a different set of peptides but the distribution was approximately the same, so the IEs were similar. However, when the two antibodies were mixed, the distribution of the IMS signal expanded, which in turn caused the entropy to be higher than a single antibody. This result confirms that entropy can in principle be used as a measure of the disorder in an IMS.

IE Varies with Gender, Blood Type, and Ethnicity but not Age or Location

In order to identify factors associated with IE, the sera of 800 healthy individuals was examined using the IMS platform. These samples were obtained from Clinical Testing Solutions (CTS Inc., Tempe, Ariz.) and were chosen to equally represent the proportion of genders, ethnicity, blood types, and ages in the Southwest US population. They were collected from centers in California, Arizona and Texas.

In FIG. 2 the distribution of entropy values across the whole set of 800 samples is presented. The entropy values ranged from 6.6 to 8.8 with a median of 8.1. The values are approximately normally distributed.

FIG. 2 shows the IE distribution with various factors including age, location, gender, blood type, and ethnicity. The distribution in every group follows a near normal distribution. Whether there were any significant differences in pairwise comparisons of the entropy with regard to these factors was investigated. None were found with respect to age and location. However, it was found that that the entropy values are influenced by gender, blood type, and ethnicity.

Generally, females have slightly higher entropy than males. Caucasians had a lower entropy level than Asian or African-Americans. The difference of these two sets of comparisons were at a significance level of <0.005 by a t-Test and <0.0001 by an ANOVA test.

Differences in IE both in the ABO blood group system and the Rh blood group system were found. People with AB blood type have on average the lowest entropy value, whereas the other blood types are similar to each other. The Rh blood system also shows that Rh− blood type has lower entropy compared with Rh+ blood type.

As noted the Caucasian and Asian populations had different average entropy levels and Rh+ and Rh− have different average values. Caucasians have a frequency of 17% for Rh− while Asians have a frequency of <2%. Given these differences, whether the differences in ethnic backgrounds could be accounted for by Rh differences was investigated. The Rh− samples were subtracted from the Asian and Caucasian derived samples and reanalyzed. The difference in entropy averages was not affected. Therefore, it appears the differences at least between the Asian and Caucasian groups is not due to differences in Rh factor.

The Entropy Value Varies Between Individuals, in the Same Individual Over Time, and can Reflect Health Status

One would assume that the entropy value between individuals would be different even if just due to random fluctuations in the immune system. However, it is not known what the range of the variation is and how it differs from person to person. In this experiment, the IE of 5 individuals over a period of time was obtained. Blood was drawn daily for 1 month and every week for 2 subsequent months, the IE determined for each sample. The variance for each individual is summarized in a box plot in FIG. 3A. An ANOVA test shows a p-value<0.0001, indicating there is significant difference in the mean entropy for the 5 individuals. This indicates that random fluctuations alone are not sufficient to explain the difference between individuals. It is interesting to note that people with lower average entropy tend to have lower variation. The standard error correlates well with the average entropy value. This is especially the case for volunteers 4 and 5, both of whom had the lowest average entropy and variance.

How entropy changes over time within an individual and between them was investigated. Instead of plotting the entropy values in a boxplot graph, the entropy change with time in each of the individuals was illustrated in FIG. 3B. Five volunteers are monitored during the same time period. As it shown, the entropy for all individuals varies during this period and does not show a time correlation between individuals. It appears that the variance in entropy is quite different between individuals.

To determine whether entropy can truly reflect the health status of an individual, the volunteers' health and vaccine history was recorded during the monitored time period. An example of one individual is graphed in FIG. 3C. Volunteer 4 received 3 vaccines, and was self-reported sick during the monitoring period. Aside from the missing data points from July 25^{th} to early August, there was a trend for the entropy value to increase on health events. This gives us a first indication that entropy can be used to monitor health status as it changes with exposure to infections or vaccines.

Entropy is Higher for People Infected with Pathogens

Once it was established how entropy changes in healthy individuals, it was asked whether entropy value changes with different forms of health disturbance. This was first tested with infectious diseases. Sera from seven types of infections were assayed, including Borrelia, Bordetella pertussis, dengue, Hepatitis B virus, malaria, syphilis and West Nile Virus. All samples were from convalescent people. These pathogens, including bacterial, viral and parasite infections, were chosen to broadly reflect the infectious population.

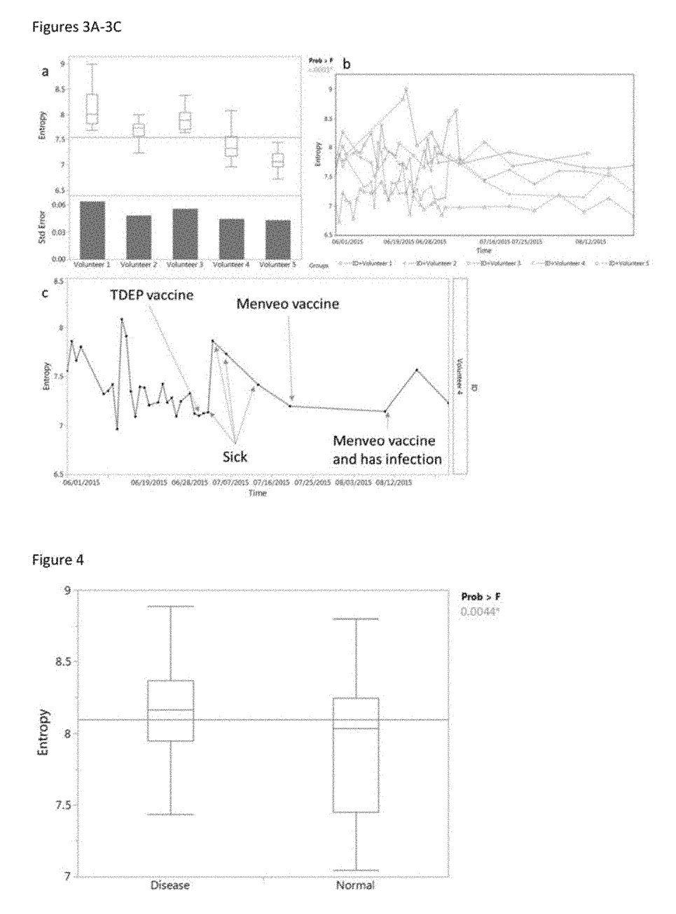

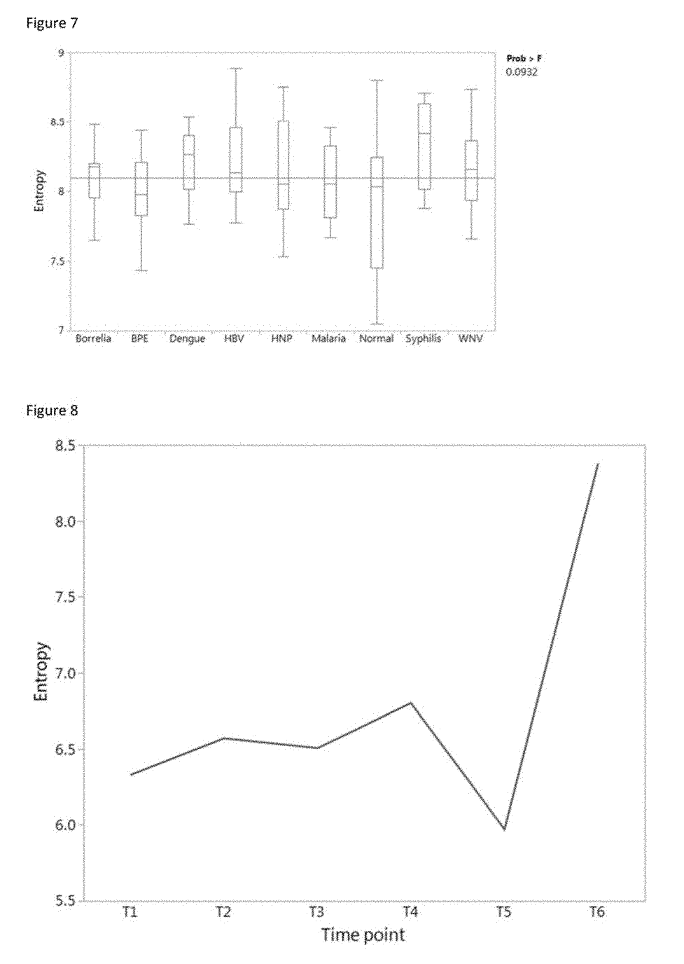

When comparing them with non-infected samples, the infection group shows significantly higher entropy level (FIG. 4). This result implies that entropy can indeed distinguish people with different health status. Result of the un-mixed 7 pathogens' entropy comparison is attached in FIG. 7.

Sera from People with Cancer Exhibited a Higher Level of Entropy

If people with cancer have differences in average entropy was tested. Cancer signatures are distinct by type and from infections (Hanahan, D. & Weinberg, R. A. Hallmarks of cancer: the next generation. Cell 144, 646-674, doi:10.1016/j.cell.2011.02.013 (2011); Hanahan, D. & Weinberg, R. A. The hallmarks of cancer. Cell 100, 57-70 (2000)). A tumor presumably presents more antigens, including neo-antigens, to the immune system and is often subject to immune suppression (Kawakami, Y. et al. Identification of human tumor antigens and its implications for diagnosis and treatment of cancer. Cancer science 95, 784-791 (2004); Andersen, R. S. et al. Dissection of T-cell antigen specificity in human melanoma. Cancer research 72, 1642-1650, doi:10.1158/0008-5472.CAN-11-2614 (2012); Reiman, J. M., Kmieciak, M., Manjili, M. H. & Knutson, K. L. Tumor immunoediting and immunosculpting pathways to cancer progression. Seminars in Cancer Biology 17, 275-287 (2007); Whiteside, T. L. Immune suppression in cancer: effects on immune cells, mechanisms and future therapeutic intervention. Seminars in cancer biology 16, 3-15, doi:10.1016/j.semcancer.2005.07.008 (2006)).

Here, datasets from normal donors and from people with three types of cancer (breast cancer, lung cancer and multiple myeloma) were used to represent general cancer patients. The sample sizes were balanced in each group, with ˜20 cancer and ˜20 healthy donors. As shown in FIG. 5A, cancer samples have significantly higher entropy value compared with healthy donors. The P-value from T-Tests is <0.0007.

In some B-cell lymphomas, a large amount of the same antibody is produced, which changes the antibody composition in the blood (Shaffer, A. L., 3rd, Young, R. M. & Staudt, L. M. Pathogenesis of human B cell lymphomas. Annual review of immunology 30, 565-610, doi:10.1146/annurev-immunol-020711-075027 (2012); Kuppers, R. Mechanisms of B-cell lymphoma pathogenesis. Nat Rev Cancer 5, 251-262, doi:10.1038/nrc1589 (2005)). It is predicted that this may lead to lower entropy value compared with healthy donors. To test this prediction, the IMS was determined for dogs with a B-cell lymphosarcoma (LSA) to healthy dogs. IMS uses the same chip for all diseases and species, just requiring the appropriate, in this case dog, secondary, labeled antibody. 68 normal dogs were compared to 83 LSA samples. As evident the entropy is significantly lower in the LSA compared with healthy dogs. This is consistent with the prediction.

A much larger population monitoring study funded by the Department of Homeland Security was conducted. 200 volunteers gave a blood sample every two weeks for 4 months. They self-provided the sample on a blood card from a finger stick or a Tasso, self-collection device. Some of the individuals volunteered to receive a tetanus vaccine during the trial. All samples were assessed on 125K peptide arrays for their IE.

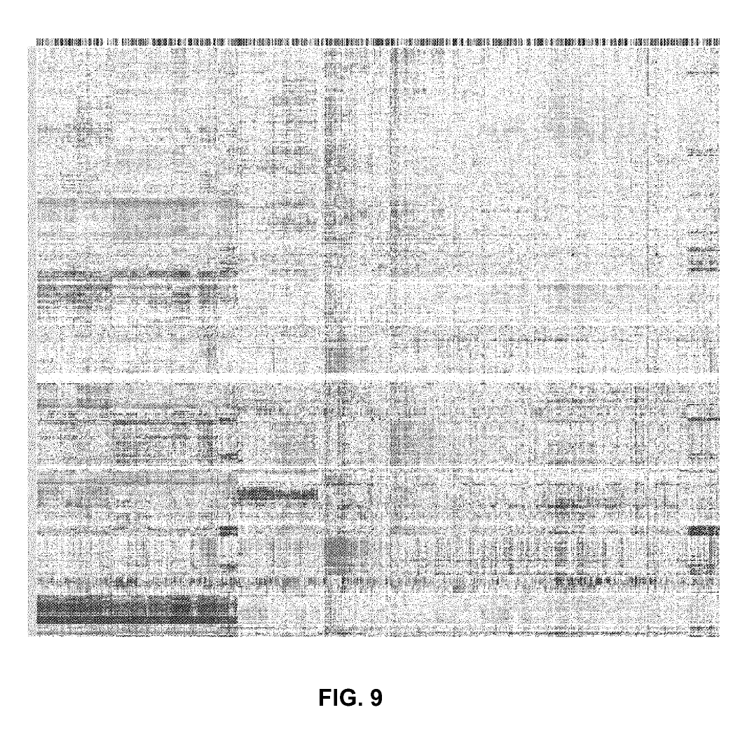

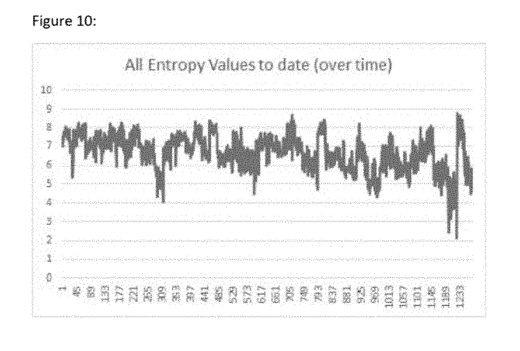

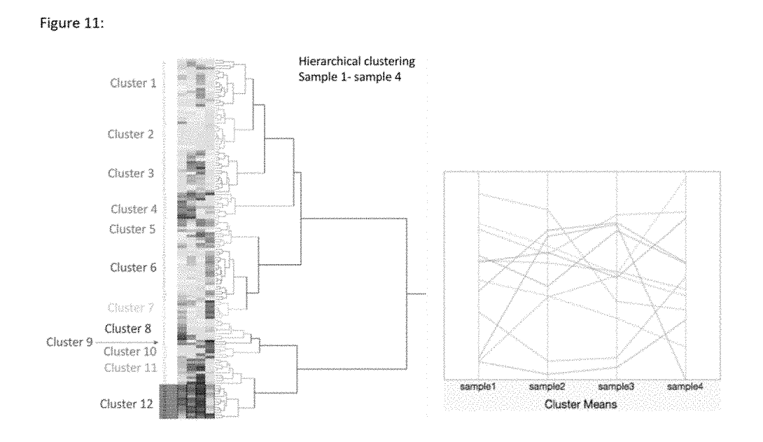

In FIG. 9, a portion of the complexity of the signatures gathered from the individuals is depicted. In FIG. 10, it is demonstrated that this complexity can be reduced to single IE numbers that can be plotted to exhibit patterns—decline in IE after vacations. In FIG. 11, it is demonstrated that by representing the immune status as IE other comparisons and plottings can be generated.

Discussion

The application of Shannon information entropy to monitoring immune health was explored. It was shown that two different monoclonal antibodies that bind to a different set of peptides and have comparable entropy measures, produce an increase in entropy when mixed and added to the arrays, as predicted. A collection of sera from 800 people who equally represent gender, age, ethnic background and three geographic locations was used to measure the entropy of IMS for each. It was found that the entropy values ranged from ˜6.6 to 8.8 and were approximately normally distributed over the 800 samples. In pairwise comparison of various sets of signatures, it was found that there were no significant differences in average entropy values between age or geographic location. The average values females were slightly higher than males, and Asian and African-American donors were significantly higher than that of Caucasian donors. While there were no differences in averages between A, B and O blood types, AB blood types were significantly lower on average. Rh− samples were on average lower than Rh+. The difference between Asian and Caucasian donor samples could not be explained by differences on Rh− frequency between the two groups. The analysis was extended to samples from people infected with 7 different pathogens and found that as a pool these samples had on average significantly higher entropy values than uninfected controls. The same was true for samples from people with three different cancers compared to people without cancer. However, dogs with a B-cell lymphoma, as might be predicted for a clonal production of a particular antibody, actually had lower average entropy levels. This approach was practical for monitoring health on a regular basis in a study with 200 people monitored every two weeks over 4 months. Changes in individuals over time, group average over time and in response to vaccination were noted.

In the proof of principle experiment, two different high affinity monoclonal antibodies to two different sites on P53 were used (FIG. 1). Monoclonal antibodies can vary greatly in the number of peptides they bind in the array (Halperin, R. F., Stafford, P., Legutki, J. B. & Johnston, S. A. Exploring antibody recognition of sequence space through random-sequence peptide microarrays. Molecular and Cellular Proteomics 28, e101230.101236 (2010)). It is suggested that the entropy assessment of an antibody may be a good predictor of off-target binding. It would have the value of being a simple, single number standard that could be applied to all antibodies. It may be useful for evaluation of antibodies or aptamers for therapeutics, purification or research uses.

While there was a wide range of entropy values in each of the groups in the 800 samples (FIG. 2), there were significant differences in the average for gender, ethnicity, and blood groups (FIG. 2). The underlying causes of these differences is unknown. Given that the immune system is highly sensitive to both intrinsic and extrinsic factors it would take more studies to associated a cause(s) of the differences. Where there are no significant differences, for example geographic location, differences in flora, for example, can be excluded as inducing different average entropy levels. These differences were unexpected and could only have been discovered by applying this technique.

Five people were monitored daily for one month and then weekly for an addition two months (FIGS. 3A-3C). This allowed us to determine the differences in averages overtime and the variance for each person over time. The entropy averages of the 5 people happened to represent approximately the range observed in the 800 samples. Each person generally maintained the differences between each other over the three months. The person with the highest average entropy also had the highest variance and the one with the lowest the lowest variance. It will be interesting to see in a larger set of individuals whether this generally holds true. In order to see if a health event changed the entropy value of an individual, one person received a vaccine. There was subsequently a sharp increase in the entropy number for this individual (FIG. 3C), although the increase was within the range they previously presented. Additionally, one individual later had an undiagnosed illness and this was accompanied by an increase in entropy (FIG. 7). These are single events so the association between entropy increase and illness could be coincidental.

The results of the monitoring of individuals suggests two potential applications for entropy monitoring. On an individual level if a person monitors their entropy over time on a regular basis, one could detect a significant change from baseline or normal variance. To be useful this would change would need to be present before symptoms occurred. Whether entropy changes are present before symptoms is another area of future investigation.

Another potential application would be for population monitoring for a disease outbreak or an intentional biological attack. If a population was monitoring their IE on a regular basis, presumably in order to detect early signs of a chronic disease, a disturbance in the entropy levels of a large number of people could be an indicator of an event. As evident from the data in FIGS. 3A-3C on monitoring individuals, this would need to be based on multiple measures of time of each individual. It may be possible to identify the peptides that were responsible for the change in entropy in each person and determine if there was a common basis for the alteration. In the case of a natural outbreak or attack, this signature would represent the immune response to the infectious agent.

The practicality of monitoring a population over time was demonstrated in a study of 200 people. Individuals sent in a blood sample (two methods were tested) they collected themselves (FIGS. 9,10,11). These samples were monitored in a timely fashion. An infectious disease outbreak could theoretically have been detected.

In the data presented in FIGS. 3C and 7, the disturbance health event was accompanied by an increase in entropy. Whether this is generally the case was investigated. For both infections (FIG. 4) and cancers (FIG. 5A), the people with the health problem had on average higher entropy levels. However, within both diseases there was a wide range in entropy values for different people. Therefore, even for a health disturbance that causes and increase in entropy, it would need to be measured against the personal baseline. As an example of entropy decreasing, an analysis of dogs diagnosed with a B-cell lymphosarcoma (LSA) was presented. In contrast to the data in FIG. 5B, the average entropy was lower in the disease state. B-cell cancers may be a special case as they are characterized by overproduction of one antibody species.

Infections induce a set of high affinity antibodies to the pathogen. In order for this to register as an increase in entropy the induced antibodies would need to expand the number of sites bound relative to the peptides bound by the non-infected samples. The implication is that there would need to be unoccupied features that the induced antibodies could bind to expand the diversity. Presumably, this would also be the case for the cancer samples. In the case of the LSA samples the preponderance of the antibody produced by the cancerous B-cell would decrease the total diversity of antibodies in the sample to lead to a decrease in average entropy.

As discussed in the Introduction, the concept of entropy has been applied to various measures of the immune system. The approach of sequencing B-cell variable regions in depth most closely resembles our concept. For example, Asti et al. used deep sequencing data on HIV patients as applied to predict binding to HIV antigens (Asti, L., Uguzzoni, G., Marcatili, P. & Pagnani, A. Maximum-Entropy Models of Sequenced Immune Repertoires Predict Antigen-Antibody Affinity. PLoS computational biology 12, e1004870, doi:10.1371/journal.pcbi.1004870 (2016)). Using IE to measure entropy of the antibody repertoire has several advantages. The blood spots for the IMS analysis can be sent through regular mail and only requires a small amount of blood, making large population surveys feasible (Chase, B. A., Johnston, S. A. & Legutki, J. B. Evaluation of biological sample preparation for immunosignature-based diagnostics. Clinical and Vaccine Immunology 19, 352-358 (2012)). The assay itself is simple and inexpensive. The simplicity of this approach to measuring the humoral immune component should encourage further investigations and applications.

Material and Methods

Array Platforms

Two different immunosignature peptide array platforms were used: two different libraries of 10,000 peptide microarrays, the CIM10Kv1 (NCBI GEO accession number pending), the CIM10Kv2 (GPL17600) and HT330K (GPL17679). The 10K random peptide platforms consists of 10K 20 residue peptides linked to glass slides through a maleimide conjugation to a linker coupled to an aminosilane-coated glass surface. This linker is on the carboxyl terminus for CIM10Kv1 and on the amino terminus for CIM10Kv2 (Stafford, P. et al. Physical characterization of the ‘Immunosignaturing Effect’. Molecular & Cellular Proteomics, doi:10.1074/mcp.M111.011593 (2012)). The CIM10Kv1 arrays were produced by spotting peptides synthesized by Alta Biosciences using a NanoPrint LM60 microarray printer (Arrayit, Sunnyvale, Calif.). The CIM10Kv2, peptides were synthesized by Sigma Genosys (St. Louis, Mo.), and they were printed by Applied Microarrays (Tempe, Ariz.) using a piezo non-contact printer.

The 330K platform (GPL17679) uses an in situ synthesis method to create 330,000 peptides on a silicon wafer (Legutki, J. B. et al. Scalable high-density peptide arrays for comprehensive health monitoring. Nature communications 5 (2014)). This platform uses peptides selected from random space to maximally distribute the peptides in that space. On this platform, not all of the peptides have exactly the same length, but average 12 amino acids plus or minus 6 amino acids at the 95^{th} percentile. Arrays are deprotected following synthesis, soaked overnight in dimethyl formamide. The residual DMF was removed by two 5 min washes in distilled water, then arrays are soaked in PBS pH 7.3 for 30 min, blocked with an incubation buffer (3% BSA in Phosphate Buffered Saline, 0.05% Tween 20 (PBST)), washed, and spun dry, 1500 RPM×5′. At this point the, the arrays were ready for the application of sera.

Array Procedures with Samples

The general assay conditions have been published previously, and briefly described here (Halperin, R. F., Stafford, P., Legutki, J. B. & Johnston, S. A. Exploring antibody recognition of sequence space through random-sequence peptide microarrays. Molecular and Cellular Proteomics 28, e101230.101236 (2010); Brown, J., Stafford, P., Johnston, S. & Dinu, V. Statistical methods for analyzing immunosignatures. BMC Bioinformatics 12, 349 (2011); Kukreja, M., Johnston, S. A. & Stafford, P. Immunosignaturing microarrays distinguish antibody profiles of related pancreatic diseases. Proteomics and Bioinformatics S6, doi:doi:10.4172/jpb.S6-001 (2012); Kukreja, M., Johnston, S. A. & Stafford, P. Comparative study of classification algorithms for immunosignaturing data. BMC Bioinformatics 13, doi:doi:10.1186/1471-2105-13-139 (2012)). The procedure for applying sample to the arrays of the two different types of platforms is nearly identical, and less than 1 μl of sample is required. For the CIM10K platform, the microarrays are pre-washed in 10% acetonitrile, 1% BSA to remove unbound peptides. Then the slides are blocked with 1×PBS pH 7.3, 3% BSA, 0.05% Tween 20, 0.014% β-mercaptohexanol for 1 hr RT. Without drying, slides are immersed in sample buffer consisting of 3% BSA, 1×PBS, and 0.05% Tween 20 pH 7.2. Serum is diluted 1:500 and applied to the peptide array for 1 hr at 37° C. The slides are washed in 1× Tris-buffered saline with 0.05% Tween 20 (TBST) pH 7.2. Then a mouse anti-human secondary antibody conjugated to a dye is applied to the array. The slides are washed again as before and dried by centrifugation. The slides are then scanned in an Agilent ‘C’ scanner to determine the intensity of each peptide. For the 330 k platform, the arrays were loaded into a multi-well Array-It gasket. Then a volume of 100 μl of incubation buffer was added to each well, and then 100 μl of 1:2,500 diluted sera was added for a final concentration of 1:5,000. Arrays were incubated for 1 hr at room temperature (RT) with rocking, and then washed with PBST using a BioTek 405TS plate washer. An anti-human IgG-DyLight 549 secondary antibody with a conjugated dye (KPL, Gaithersburg, Md.) was added to the sera at a final concentration of 5 nM. This solution was incubated 1 hr at RT with rocking, and unbound secondary was then removed with PB ST followed by distilled water. The arrays were removed from the gasket while submerged, dunked in isopropanol, and centrifuged dry at 800×g for 5 min. These arrays were then scanned with a commercially available scanner to determine the intensity of a certain wavelength at each peptide feature position.

Once the 16 bit TIFF image file from either type of array was obtained, the intensity values from each feature were obtained using GenePix 8.0 (Molecular Devices, Santa Clara, Calif.). These fluorescence intensity values were then used to calculate the value of global measures such as the mean and Shannon information entropy.

Java Entropy Program

A custom Java program was written to calculate Shannon's entropy from the fluorescence intensity files (.gpr, or “Gene Pix Array Format”) from the peptide microarray. Most image alignment software allows output as a gpr file, and that is how the program recognizes data columns. However, any datatype could be used with minor modifications. There are two programs listed in the herein, an algorithm class and a test class. The algorithm class provides values entropy given an immunosignature data file, but for comparison sake it also provides CV (coefficient of variance), mean, median, kurtosis, skew, 95^{th} percentile, 5^{th} percentile, and dynamic range. Tests have shown that entropy is the most sensitive and robust to health changes, but the other calculations provide comparisons. The test class allows the user to input their data directories and filenames, and serves as the Java main class.

Software and Statistics for General Analysis

Microsoft Excel and JMP were used for data analysis and to create the graphs. Linear fit of entropy on age is by ordinary least squares. P-value is the probability of aging is actually influencing entropy. Either ANOVA test or t-Test is used in testing if entropy is being influenced by specific factors.

Java MAIN (TEST) CLASS

import java.io.File;
import java.nio.file.Paths;
import java.text.DateFormat;
import java.text.SimpleDateFormat;
import java.util.*;
import java.util.regex.Pattern;
public class Test_Immunosignature_Data_030413d0955 {

```
private UsefulTools useful_tools = new UsefulTools( );
private DataPreparationClass dpc = new DataPreparationClass( );
private NormalizedDataHandler ndh = new NormalizedDataHandler( );
private CalculationHandler ch = new CalculationHandler( );
TestHandler test_handler = new TestHandler( );
ScenarioHandler sh = new ScenarioHandler( );
String gpr_data_directory = “”;
String result_data_directory = “”;
//set dye_type to “F555 Median” or “F647 Median”
String dye_type = “F647 Median”;
/**
  * @param args
  */
public static void main(String[ ] args) {
```

if(args.length>0)
{Test_Immunosignature_Data_030413d0955 tid = new

Test_Immunosignature_Data_030413d0955(args);}

else{Test_Immunosignature_Data_030413d0955 tid = new

Test_Immunosignature_Data_030413d0955( );

| | |
|---|---|
| tid.test( ); | }} |

public Test_Immunosignature_Data_030413d0955( )
public Test_Immunosignature_Data_030413d0955(String[ ] args)
{Test_Immunosignature_Data_030413d0955FromCommandLine(args);}
public Test_Immunosignature_Data_030413d0955(String directory, String filename)
{System.out.println(useful_tools.getTime( ));

ScenarioHandler sh = new ScenarioHandler( );
sh.find_summary_numbers_one_gpr(directory, filename, “F532 Median”);
System.out.println(useful_tools.getTime( ));}

public void Test_Immunosignature_Data_030413d0955FromCommandLine(String[ ] args)
{/*

```
  *
  * //-collectAllSummaryFilesIntoOneTable(String directory, String
```

name_of_summary_file, String output_file_name)

//--command line version: collectAllSummaryFilesIntoOneTable directory name_of_summary_file

output_file_name

```
  *
  *
  *
*/
String return_string = useful_tools.getTime( )+“\r\n”;
String directory = args[1];
String call_details =“”;
if(args[0].equals(“find_summary_numbers_one_gpr”))
{call_details = “find_summary_numbers_one_gpr_”+args[2];
```

if(args[4]!=null)
{sh.find_summary_numbers_one_gpr(args[1], args[2], args[3],

Integer.valueOf(args[4]).intValue( ));}else

{sh.find_summary_numbers_one_gpr(args[1], args[2], args[3]);}

else if(args[0].equals(“find_summary_numbers_from_folder_of_gprs”))
{call_details = “find_summary_numbers_from_folder_of_gprs_”+args[1];

if(args[3]!=null)
{sh.find_summary_numbers_from_folder_of_gprs(args[1], args[2],

Integer.valueOf(args[3]).intValue( ));}else

{sh.find_summary_numbers_from_folder_of_gprs(args[1], args[2]);}} else if

(args[0].equals(“find_summary_numbers_from_tabdelimitedtext_raw_data”))

{call_details = “find_summary_numbers_from_tabdelimitedtext_raw_data_”+args[1];

if(args[9]!=null)

{sh.find_summary_numbers_from_tabdelimitedtext_raw_data(args[1], args[2],

Integer.valueOf(args[3]).intValue( ), Integer.valueOf(args[4]).intValue( ),
Integer.valueOf(args[5]).intValue( ), Integer.valueOf(args[6]).intValue( ),
Integer.valueOf(args[7]).intValue( ), Integer.valueOf(args[8]).intValue( ),
Integer.valueOf(args[9]).intValue( ));}else{

sh.find_summary_numbers_from_tabdelimitedtext_raw_data(args[1], args[2],

Integer.valueOf(args[3]).intValue( ), Integer.valueOf(args[4]).intValue( ),
Integer.valueOf(args[5]).intValue( ), Integer.valueOf(args[6]).intValue( ),
Integer.valueOf(args[7]).intValue( ), Integer.valueOf(args[8]).intValue( ));}}

else if(args[0].equals(“find_summary_numbers_from_tabdelimitedtext_normalised_data”)
{call_details =

“find_summary_numbers_from_tabdelimitedtext_normalized_data_”+directory;

if(args[9]!=null)
{sh.find_summary_numbers_from_tabdelimitedtext_normalized_data(args[1], args[2],

Integer.valueOf(args[3]).intValue( ), Integer.valueOf(args{4]).intValue( ),
Integer.valueOf(args[5]).intValue( ), Integer.valueOf(args[6]).intValue( ),
Integer.valueOf(args[7]).intValue( ), Integer.valueOf(args[8]).intValue( ),
Integer.valueOf(args[9]).intValue( ));}else

{sh.find_summary_numbers_from_tabdelimitedtext_normalized_data(args[1], args[2],

Integer.valueOf(args[3]).intValue( ), Integer.valueOf(args[4]).intValue( ),
Integer.valueOf(args[5]).intValue( ), Integer.valueOf(args[6]).intValue( ),
Integer.valueOf(args[7]).intValue( ), Integer.valueOf(args[8]).intValue( ));}}

else if(args[0].equals(“collectAllSummaryFilesIntoOneTable”))
{call_details = “collectAllSummaryFilesIntoOneTable_”+directory;

sh.collectAllSummaryFilesIntoOneTable(args[1], args[2], args[3]);}

else if(args[0].equals(“placeFilesInFolderIntoTheirOwnFolder”))
{call_details = “placeFilesInFolderIntoTheirOwnFolder_”+directory;
sh.placeFilesInFolderIntoTheirOwnFolder(args[1]);}
return_string+=useful_tools.getTime( );
call_details=call_details.replaceAll(“:”, “C”);
call_details=call_details.replaceAll(Pattern.quote(File.separator),

“S”);useful_tools.createTextFile(directory, “runtime_info.txt”, return_string);}

public void test( )
{System.out.println(useful_tools.getTime( ));

TestHandler th = new TestHandler( );

{“find_summary_numbers_from_tabdelimitedtext_normalized_data”,”};
{“find_summary_numbers_from_tabdelimitedtext_normalized_data”,“YourDirectoryHere”,“2”,“1”,“2

”,“1”,“2”,“6”};
String[ ] arguments = {“find_summary_numbers_one_gpr”, “YourDirectoryHere”,“4-46 S2 F1 Hi P20 110512
ND145 50k S”,“F532 Median”, “5”};
Test_Immunosignature_Data_030413d0955FromCommandLine(arguments);
{“find_summary_numbers_from_folder_of_gprs”,“YourDirectoryHere”,“F532 Median”,“true”,“1”,“20”,“10”};
“YourDirectoryHere”, “F647 Median”, “false”, “1”, “65535”, “10000”};
System.out.println(useful_tools.getTime( ));}}

Although the foregoing specification and examples fully disclose and enable the present invention, they are not intended to limit the scope of the invention, which is defined by the claims appended hereto. Additionally, aspects of the present discoveries are included in Wang et al., Scientific Reports, 7, 18060 (2017), the disclosure of which, including Supplemental Information, is incorporated by reference.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of determining the complexity of a mixture of antibodies, characterizing the binding distribution of an antibody or aptamer, determining a Shannon immune entropy (IE) in an individual, or measuring the immune health of a subject or population by quantifying the diversity, organization and disorder of the antibodies in the subject, the method comprising:
(a) contacting a physiological sample with an array platform comprising at least 10⁴ to 10 peptides of random sequences, wherein each peptide is 6-20 amino acids long and is operably linked to a solid substrate having an area of about 0.5 cm² to 2.5 cm² to form a sample-coated array platform,
(b) contacting the sample-coated array platform with a labeled binding agent that binds to the sample, and
(c) measuring an intensity distribution of the label.

2-3. (canceled)

4. The method of claim 1, wherein the method further comprises calculating the Shannon IE of feature fluorescence.

5. The method of claim 1, wherein the binding agent is an antibody, dye, or aptamer.

6. The method of claim 1, wherein the label is a dye, fluorescent label, quantum dot or gold nanosphere.

7. (canceled)

8. The method of claim 1, wherein the binding agent is measured by mass spectrometry.

9. The method of claim 1, wherein the quantification is Shannon's entropy of the binding agents to the sample-coated array platform.

10. A method of determining an immune entropy (IE) value, the method comprising:
(a) applying a physiological sample or purified antibody or aptamer to an array platform comprising at least 10⁴ to 10⁸ peptides of random sequences, wherein each peptide is 6-20 amino acids long operably linked to a solid substrate having an area of about 0.5 cm² to 2.5 cm²,
(b) pre-washing the platform to remove unbound peptides,
(c) blocking the platform with a blocking solution,
(d) immersing the platform in sample buffer,
(e) diluting a subject's serum sample at least 1:500 and applying the diluted sample to the platform,
(f) washing the platform in a second wash solution,
(g) applying to the platform an anti-human secondary antibody conjugated to a dye,
(h) washing the platform, and
(i) scanning the platform to determine the intensity of the dye.

11. The method of claim 10, wherein in step (b) the platform is pre-washed with 10% acetonitrile, 1% BSA.

12. The method of claim 10, wherein in step (c) the blocking solution is 1×PBS pH 7.3, 3% BSA, 0.05% Tween 20, 0.014% β-mercaptohexanol.

13. The method of claim 10, wherein in step (d) the sample buffer comprises 3% BSA, 1×PBS, and 0.05% Tween 20 pH 7.2.

14. The method of claim 10, wherein in step (f) the second wash solution comprises 1× Tris-buffered saline with 0.05% Tween 20 (TBST) pH 7.2.

15. The method of claim 10, wherein the solid substrate is glass, silicone, quartz or other form of slide.

16. The method of claim 10, wherein the solid substrate is coated with aminosilane, nitrocellulose, epoxy, dendrimers, or other platform for attachment of peptides.

17. The method of claim 16, wherein the peptides are operably linked by means of maleimide conjugation to a linker, and wherein the linker is operably linked to the aminosilane-coated glass.

18. A method of determining an immune entropy (IE) value, the method comprising:
(a) loading a platform comprising at least 10⁴ to 10⁸ peptides of random sequences, wherein each peptide is 6-20 amino acids long operably linked to a well in a multi-well gasket,
(b) adding a volume of 1-100 μl of incubation buffer to each well in the platform,
(c) diluting a physiological sample at least 1:50 to 1:500 and applying the diluted sample to the plurality of wells in the platform,
(d) washing the platform with a wash solution,
(e) applying to the plurality of wells in the platform an anti-human secondary antibody conjugated to a dye,
(f) washing the platform, and
(g) scanning the platform to determine the intensity of the dye.

19. The method of claim 18, wherein in step (d), the platform is washed using a BioTek 405TS plate washer.

20. The method of claim 18, wherein in step (d), the wash solution is 3% BSA in Phosphate Buffered Saline, 0.05% Tween 20 (PBST).

21. The method of claim 18, wherein the physiological sample is blood, serum, plasma or saliva.

22. The method of claim 18, wherein the array platform comprises 10⁴ to 3×10⁵ peptides.

23. A method for determining a difference in distribution of two immune entropy (IE) datasets relating to a subject comprising (a) calculating a first IE dataset value for an individual using the method of claim 18, (b) calculating a second IE dataset value for the individual using the method of claim 18, and (c) determining the change in IE dataset values.

24. The method of claim 23, wherein the second data set is calculated from a sample taken from the patient at least one day later than the first sample.

25. The method of claim 23, wherein the second data set is calculated from a sample taken from the patient at least one week later than the first sample.

26. The method of claim 23, wherein the second data set is calculated from a sample taken from the patient at least one month later than the first sample.

27. A method of monitoring a population for disease outbreak comprising:
(a) determining a first IE value of a plurality of individuals in a population at a first time point using the method of claim 18,
(b) determining a second IE value of a plurality of individuals in a population at a second time point using the method of claim 18, and
(c) comparing the first and second IE values to determine the change in immune entropy.
